(19) United States
(12) Patent Application Publication
(10) Pub. No.: US 2025/0000661 A1
(43) Pub. Date: Jan. 2, 2025

(54) POSTERIOR-STABILIZED ORTHOPAEDIC INSERT AND SYSTEM

(71) Applicant: DePuy Ireland Unlimited Company, Ringaskiddy (IE)

(72) Inventors: Luke J. Aram, Winona Lake, IN (US); Michael J. Rock, Leeds (GB); Travis D. Bennett, Huntington, IN (US); Stephen E. White, Fort Wayne, IN (US); Amitkumar M. Mane, South Whitley, IN (US)

(21) Appl. No.: 18/754,490

(22) Filed: Jun. 26, 2024

Related U.S. Application Data
(60) Provisional application No. 63/524,113, filed on Jun. 29, 2023.

(51) Int. Cl. A61F 2/38; A61F 2/30

(52) U.S. Cl. CPC A61F 2/389; A61F 2/30734; A61F 2002/30136; A61F 2002/30331; A61F 2002/30878

(57) ABSTRACT

An orthopaedic knee prosthesis includes a femoral component and a tibial insert including a post. The femoral component is configured to articular on the tibial insert. The post of the tibial insert includes features that facilitate the medial pivoting of the femoral component through a range of flexion.

The present application claims priority under 35 U.S.C. §119 to, and the benefit of, U.S. Provisional Patent Application Ser. No. 63/524,113, entitled “POSTERIOR-STABILIZED ORTHOPAEDIC SYSTEM FOR MEDIAL PIVOTING OF FEMORAL COMPONENT,” which was filed on Jun. 29, 2023, the entirety of which is expressly incorporated herein by reference.

### TECHNICAL FIELD

The present disclosure relates to orthopaedic knee prosthesis systems and, more specifically, to posterior-stabilized orthopaedic knee prostheses and methods for total knee arthroplasty procedures.

### BACKGROUND

Joint arthroplasty is a well-known surgical procedure by which a diseased and/or damaged natural joint is replaced by a prosthetic joint. A typical knee prosthesis includes a tibial tray, a femoral component, a patella component, and a polymer insert or bearing positioned between the tibial tray and the femoral component. Depending on the severity of the damage to the patient's joint, orthopaedic prostheses of varying mobility may be used. For example, the knee prosthesis may include a “fixed” tibial insert in some cases wherein it is desirable to limit the movement of the knee prosthesis, such as when significant soft tissue damage or loss is present. Alternatively, the knee prosthesis may include a “mobile” tibial insert in cases wherein a greater degree of freedom of movement is desired. Additionally, the knee prosthesis may be a total knee prosthesis designed to replace the femoral-tibial interface of both condyles of the patient's femur or a uni-compartmental (or uni-condylar) knee prosthesis designed to replace the femoral-tibial interface of a single condyle of the patient's femur.

The type of orthopedic knee prosthesis used to replace a patient's natural knee may also depend on whether the patient's posterior cruciate ligament is retained or sacrificed (i.e., removed) during surgery. For example, if the patient's posterior cruciate ligament is damaged, diseased, and/or otherwise removed during surgery, a posterior-stabilized knee prosthesis may be used to provide additional support and/or control at later degrees of flexion.

Typical orthopaedic knee prostheses are generally designed to duplicate the natural movement of the patient's joint. As the knee is flexed and extended, the femoral and tibial components articulate and undergo combinations of relative anterior-posterior motion and relative internal-external rotation. However, the patient's surrounding soft tissue also impacts the kinematics and stability of the orthopaedic knee prosthesis throughout the joint's range of motion. That is, forces exerted on the orthopaedic components by the patient's soft tissue may cause unwanted or undesirable motion of the orthopaedic knee prosthesis. For example, the orthopaedic knee prosthesis may exhibit an amount of unnatural (paradoxical) anterior translation as the femoral component is moved through the range of flexion.

### SUMMARY

According to one aspect, an orthopaedic insert comprising may include a platform and a post extending superiorly from the platform. The platform may include a lateral articular surface configured to articulate with a lateral condyle of a femoral component and a medial articular surface configured to articulate with a medial condyle of the femoral component. In some embodiments, the medial articular surface may be asymmetrically shaped relative to the lateral articular surface. The post may be located between the lateral and medial articular surfaces. The post may include a posterior surface, an anterior surface opposite the posterior surface, a lateral sidewall extending from the posterior surface to the anterior surface, and a medial sidewall opposite the lateral sidewall and extending from the posterior surface to the anterior surface. The medial and lateral side walls may be concave when viewed in a coronal cross-sectional plane.

In some embodiments, a medial-lateral bisecting plane of the post may be offset from a medial-lateral bisecting plane of the platform. For example, the medial-lateral bisecting plane of the post may be offset from the medial-lateral bisecting plane of the platform in the lateral direction.

Additionally, in some embodiments, the medial and lateral side walls may be also concave when viewed in a sagittal cross-section plane. Additionally, each of the lateral and medial side walls may include a concave section and an upper end located superiorly to the respective concave section. The upper end of the medial sidewall may define a vertical plane and the upper end of the lateral sidewall is angled relative to the vertical plane defined by the medial sidewall.

In some embodiments, an angle defined between a medial-lateral bisecting plane of the platform and a first plane tangent to a first point located on the lateral side wall may be less than an angle defined by the medial-lateral bisecting plane of the platform and a second plane tangent to a second point located on the medial sidewall. In such embodiments, the selected the first point and second point may be equidistant from a bottom surface of the platform.

Furthermore, in some embodiments, the lateral articular surface may include a lateral dwell point that defines a distal-most point on the lateral articular surface and the medial articular surface may include a medial dwell point that defines a distal-most point on the medial articular surface. Additionally, a posterior-most point on the posterior surface of the post may be located posteriorly of the lateral dwell point and of the medial dwell point.

In some embodiments, the lateral articular surface may include an anterior lateral lip and a lateral dwell point that defines a distal-most point on the lateral articular surface. An inferior-superior distance between the lateral dwell point and a superior-most point of the anterior lateral lip may define a lip height of the anterior lateral lip. Similarly, the medial articular surface may include an anterior medial lip and a medial dwell point that defines a distal-most point on the medial articular surface. An inferior-superior distance between the medial dwell point and a superior-most point of the anterior medial lip may define a lip height of the anterior medial lip. In such embodiments, the lip height of the anterior medial lip may be greater than the lip height of the anterior lateral lip.

Additionally, in some embodiments, the posterior surface and each of the lateral sidewall and the medial sidewall may form a pair of posterior corners of the post. Similarly, the anterior surface and each of the lateral sidewall and the medial sidewall may form a pair of anterior corners of the post. The anterior corners may have a greater radius of curvature than the posterior corners. In some embodiments, the posterior surface of the post may have an “S-shaped” coronal cross-section that includes a concave section and a convex section that is superior to the concave section. Additionally, in some embodiments, the medial articular surface and the medial condyle of the femoral component may be more confirming to each other than the lateral articular surface and the lateral condyle of the femoral component. Furthermore, in some embodiments, the medial and lateral side walls are also concave when viewed in a transverse cross-sectional plane that bisects the post.

According to another aspect, an orthopaedic knee prosthesis may include a tibial insert having a platform and a post extending superiorly form the platform. The platform of the tibial insert includes a lateral articular surface configured to articulate with a lateral condyle of a femoral component and a medial articular surface configured to articulate with a medial condyle of the femoral component. The medial articular surface may be asymmetrically shaped relative to the lateral articular surface. In some embodiments, an angle defined between a medial-lateral bisecting plane of the platform and a first plane tangent to a first point located on the lateral side wall may be less than an angle defined by the medial-lateral bisecting plane of the platform and a second plane tangent to a second point located on the medial sidewall, wherein the first point and second point are equidistant from a bottom surface of the platform.

In some embodiments, the post may be located between the lateral and medial articular surfaces and may include posterior surface, an anterior surface opposite the posterior surface, a lateral sidewall extending from the posterior surface to the anterior surface, and a medial sidewall opposite the lateral sidewall and extending from the posterior surface to the anterior surface. The medial and lateral side walls of the post may be concave when viewed in a coronal cross-sectional plane.

Additionally, in some embodiments, the orthopaedic knee prosthesis may further includes a femoral component having a lateral condyle and a medial condyle. In such embodiment, each of the lateral condyle and the medial condyle includes a femoral articular surface defined by a plurality of curved femoral surface sections that includes a first curved femoral surface section defined by a continually decreasing radius of curvature.

In some embodiments, the lateral articular surface of the tibial insert may include a lateral dwell point that defines a distal-most point on the lateral articular surface. A contact point between the femoral articular surface of the lateral condyle of the femoral component and the lateral articular surface of the tibial insert may lie on the lateral dwell point of the lateral articular surface of the tibial insert when the femoral component is positioned in 0 degrees of flexion. Additionally, in some embodiments, the contact point between the femoral articular surface of the lateral condyle of the femoral component and the lateral articular surface of the tibial insert may move posteriorly relative to the lateral dwell point of the lateral articular surface of the tibial insert as the femoral component is moved through a range of flexion. For example, the contact point between the femoral articular surface of the lateral condyle of the femoral component and the lateral articular surface of the tibial insert is may be posteriorly of the lateral dwell point of the lateral articular surface of the tibial insert by more than 0.5 millimeters when the femoral component is positioned at 90 degrees of flexion.

In some embodiments, a medial-lateral bisecting plane of the post may be offset from a medial-lateral bisecting plane of the platform. Additionally, in some embodiments, each of the lateral and the medial side walls may include a concave section and an upper end located superiorly to the respective concave section. The upper end of the medial sidewall may define a vertical plane and the upper end of the lateral sidewall that is angled relative to the vertical plane defined by the medial sidewall.

Furthermore, in some embodiments, the lateral articular surface may include a lateral dwell point that defines a distal-most point on the lateral articular surface, and the medial articular surface includes a medial dwell point that defines a distal-most point on the lateral articular surface. In such embodiments, a posterior-most point on the posterior surface of the post may be located posteriorly of the lateral dwell point and of the medial dwell point.

Additionally, in some embodiments, the lateral articular surface may include an anterior lateral lip and a lateral dwell point that defines a distal-most point on the lateral articular surface. An inferior-superior distance between the lateral dwell point and a superior-most point of the anterior lateral lip may define a lip height of the anterior lateral lip. Similarly, the medial articular surface may include an anterior medial lip and a medial dwell point that defines a distal-most point on the medial articular surface. An inferior-superior distance between the medial dwell point and a superior-most point of the anterior medial lip may define a lip height of the anterior medial lip. In such embodiments, the lip height of the anterior medial lip may be greater than the lip height of the anterior lateral lip.

### BRIEF DESCRIPTION OF THE DRAWINGS

The detailed description particularly refers to the following figures, in which:

FIG. 1 is an exploded perspective view of an embodiment of an orthopaedic knee prosthesis;

FIG. 2 is lateral perspective view of the orthopaedic knee prosthesis of FIG. 1 in an assembled configuration;

FIG. 3 is an anterior elevation view of the orthopaedic knee prosthesis of FIG. 2;

FIG. 4 is a side elevation view of an embodiment of a femoral component of the orthopaedic knee prosthesis of FIG. 1;

FIG. 5 is a superior plan view of a tibial insert of the orthopaedic knee prosthesis of FIG. 1 illustrating a lateral arcuate articular path;

FIG. 6 is a sagittal cross-sectional view of the tibial insert of FIG. 5 taken generally along the line 6-6 of FIG. 5;

FIG. 7 is another sagittal cross-sectional view of the tibial insert of FIG. 5 taken generally along the line 7-7 of FIG. 5;

FIG. 8 is an inferior plan view of the tibial insert of FIG. 5;

FIG. 9 is an anterior elevation view of the tibial insert of FIG. 5;

FIG. 10 is a lateral side elevation view of the tibial insert of FIG. 5;

FIG. 11 is a superior plan view of a post of the tibial insert of FIG. 5;

FIG. 12 is an coronal cross-sectional view of the tibial insert of FIG. 5 taken generally along the line 12-12 of FIG. 10;

FIG. 13 is a coronal cross-sectional view of the orthopaedic knee prosthesis of FIG. 2, similar to the view of FIG. 12 taken along the line 12-12 of FIG. 10 while the femoral component is engaged with the tibial component;

FIG. 14 is another coronal cross-sectional view of the orthopaedic knee prosthesis of FIG. 13 having the femoral component undergoing an amount of lift-off from the tibial insert;

FIG. 15 is an coronal cross-sectional view of the tibial insert of FIG. 5 taken generally along the line 15-15 of FIG. 10;

FIG. 16 is a transverse cross-sectional view of the tibial insert of FIG. 5 taken generally along the line 16-16 of FIG. 10;

FIG. 17 is a sagittal cross-sectional view of the orthopaedic knee prosthesis of FIG. 2 with the femoral component at about 0 degrees of flexion;

FIG. 18 is another sagittal cross-sectional view of the orthopaedic knee prosthesis of FIG. 2 with the femoral component at about 45 degrees of flexion;

FIG. 19 is another sagittal cross-sectional view of the orthopaedic knee prosthesis of FIG. 2 with the femoral component at about 60 degrees of flexion;

FIG. 20 is another sagittal cross-sectional view of the orthopaedic knee prosthesis of FIG. 2 with the femoral component at about 90 degrees of flexion;

FIG. 21 is another sagittal cross-sectional view of the orthopaedic knee prosthesis of FIG. 2 with the femoral component at about 110 degrees of flexion; and

FIG. 22 is another sagittal cross-sectional view of the orthopaedic knee prosthesis of FIG. 2 with the femoral component at about 130 degrees of flexion.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and/or surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise. Additionally, the term “about” may be used in the specification in reference to certain measurements that are defined within manufacturing tolerances. That is, the provided measurements and/or numerical values may deviate, in practice, due to tolerances inherent in the machine or fabrication process.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

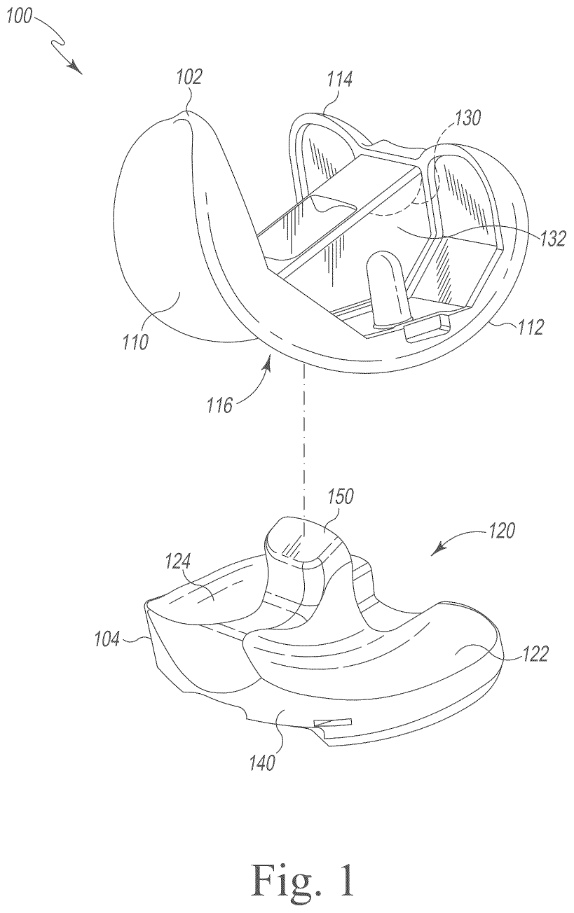

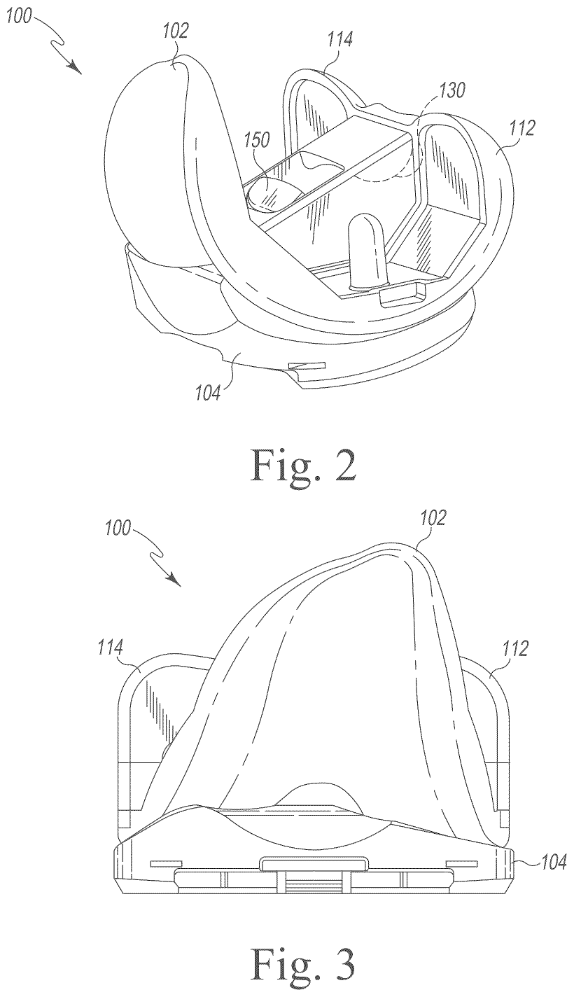

Referring now to FIGS. 1-3, in an illustrative embodiment, an orthopaedic knee prosthesis 100 includes a femoral component 102 and a tibial insert 104. Additionally, the orthopaedic knee prosthesis 100 may include a tibial tray (not shown) to which the tibial insert 104 is coupled during use. The orthopaedic knee prosthesis 100 is illustratively embodied as a posterior-stabilized orthopaedic knee prosthesis but, in other embodiments, may be embodied as other types of orthopaedic knee prostheses.

The femoral component 102 (and the tibial tray, if included) is illustratively formed from a metallic material such as cobalt-chromium or titanium, but may be formed from other materials, such as a ceramic material, a polymer material, a bio-engineered material, or the like, in other embodiments. The tibial insert 104 is illustratively formed from a polymer material such as an ultra-high molecular weight polyethylene (UHMWPE), but may be formed from other materials, such as a ceramic material, a metallic material, a bio-engineered material, or the like, in other embodiments.

The femoral component 102 is configured to be coupled to a surgically-prepared surface of the distal end of a patient's femur (not shown), and the tibial insert 104 is configured to be coupled to a surgically-prepared surface of the proximal end of a patient's tibia (not shown) via, for example, a tibial tray (not shown). Alternatively, in other embodiments, the tibial insert 104 may be configured to attach to the surgically-prepared surface of the proximal end of the patient's tibia directly, without use of a tibial tray. For example, the tibial insert 104 and a polymer “tray” may be combined into a single polymeric component.

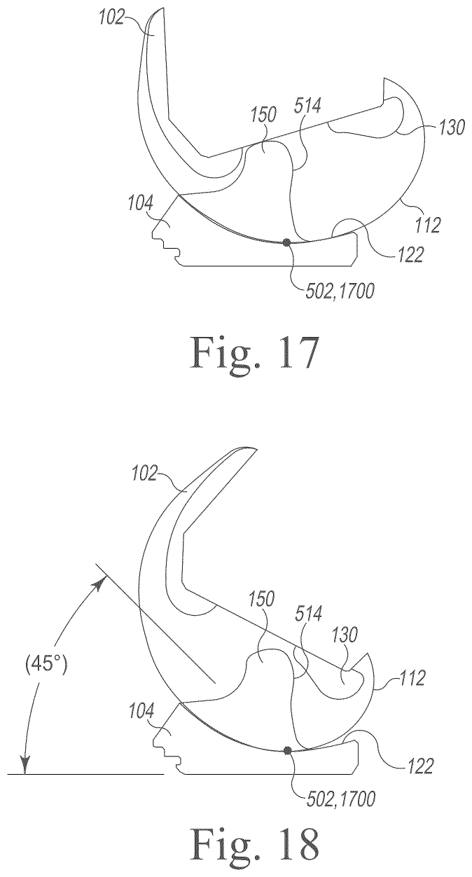

In use, the femoral component 102 is configured to articulate with the tibial insert 104. To do so, the femoral component includes an outer, articulating surface 110 having a lateral condyle 112 and a medial condyle 114. Similarly, the tibial insert 104 includes a platform 140 having an articular surface 120, which includes a lateral articular surface 122 and a medial articular surface 124. As such, the lateral condyle 112 is configured to articulate with the lateral articular surface 122, and the medial condyle 114 is configured to articulate with a medial articular surface 124 of the tibial insert as shown in FIGS. 17-22. The tibial insert 104 also includes a post 150 extending superiorly from the platform 140 and located between the lateral articular surface 122 and the medial articular surface 124. As described in more detail below, a posterior cam 130 of the femoral component 102 is configured to contact the post 150 of the tibial insert 104 during a later range of flexion.

Each of the femoral component 102 and the tibial insert 104 include articular curvatures and related features that facilitate or promote pivoting of the lateral condyle 112 on the lateral articular surface 122, while limiting or reducing anterior translation of the medial condyle 114 on the medial articular surface 124 during flexion. For example, in the illustrative embodiment, one or both of the condyles 112, 114 of the femoral component 102 includes a sagittal condylar surface having a curved surface section defined by a continuously decreasing radius of curvature. Additionally, the medial condyle 114 of the femoral component 102 and the medial articular surface 124 of the tibial insert are more conforming with each other relative to the lateral condyle 112 and the lateral articular surface 122. That is, a ratio of the coronal radius of curvature of the medial articular surface 124 to the coronal radius of curvature of the medial condyle 114 is closer to a value of 1.0 than a ratio of the coronal radius of curvature of the lateral articular surface 122 to the coronal radius of curvature of the lateral condyle 112. For example, in the illustrative embodiments, the ratio of the coronal radius of curvature of the lateral articular surface 122 to the coronal radius of curvature of the lateral condyle 112 is less than 1.0 and gradually decreases through flexion. The higher medial conformity provides stability by promoting minimal or no anterior-posterior movement, while the lower lateral confirming allows for an amount of lateral mobility and rotational freedom.

Furthermore, the post 150 of the tibial insert 104 is offset toward the medial side of the platform 140. The post 150 also includes concave sidewalls, which facilitate the medial pivoting of the femoral component 102 on the tibial insert 104 while restricting or limiting liftoff of the femoral component 102 as described in more detail below. Additionally, the lateral sidewall of the post 150 curves inwardly in the anterior direction at a greater rate than the medial sidewall of post 150 to provide additional space for the movement of the lateral condyle 112 of the femoral component. Furthermore, the anterior corners of the post 150 are rounded relative to the posterior corners of the post 150. The platform 140 of the tibial insert 104 also has an anterior medial lip height greater than an anterior lateral lip height. Each of the above-described features promote or otherwise facilitate the medial pivoting of the femoral component 102 relative to the tibial insert 104 during flexion as described in further detail below.

As discussed above, the femoral component 102 is configured to be coupled to a surgically-prepared surface of the distal end of a patient's femur (not shown) and may be secured to the patient's femur via use of bone adhesive or other attachment means. The femoral component 102 includes the lateral condyle 112 and the medial condyle 114, which are spaced apart to define an intercondylar notch or opening 116 therebetween. An intercondylar femoral box 132 is defined within the intercondylar opening 166 and includes the posterior cam 130 and may include an anterior cam (not shown) in some embodiments. In use, the condyles 112, 114 replace the natural condyles of the patient's femur and are configured to articulate on the corresponding lateral and medial articular surfaces 122, 124 of the tibial insert 104 as discussed above.

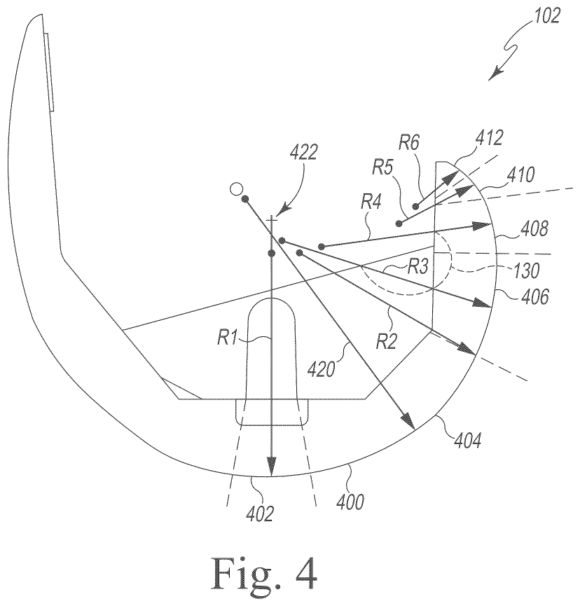

Referring now to FIG. 4, one or both of the condyles 112, 114 of the femoral component 102 include a condyle surface 400, which is convexly curved in the sagittal plane. Illustratively, the condyle surface 400 is formed from a number of curved surface sections 402, 404, 406, 408, 410, and 412 each of which is tangent to the adjacent curved surface section. Each curved surface sections 402, 404, 406, 408, 410, and 412 contacts the tibial insert 104 through different ranges of degrees of flexion. For example, the curved surface sections 402, 404 of the condyle surface 400 contact the tibial insert 104 during early flexion. The curved surface sections 406, 408 of the condyle surface 400 contact the tibial insert 104 during mid-flexion. And, the curved surface sections 410, 412 of the condyle surface 400 contact the tibial insert 104 during late flexion.

Each curved surface sections 402, 406, 408, 410, and 412 is defined by a constant radius of curvature R1, R3, R4, R5, and R6, respectively. However, as discussed in more detail below, the curved surface section 404 is defined by a plurality of rays, rather than a constant radius of curvature. In particular, the curved surface section 404 is designed to transition gradually the condyle surface 400 from the radius of curvature R1 of the curved surface section 402 to a radius of curvature R2, which is tangent to the curved surface section 406. As such, the curved surface section 402 has a continuously decreasing radius of curvature.

In other embodiments, the curved surface section 404 may be designed to provide a gradual transition from the radius of curvature R1 to the radius of curvature R2 using other geometry. For example, the radii forming the curved surface section 404 may not have common origin but may be of the same length. In such embodiments, the origin of each radii is moved along a spiral to provide a gradual transition from the radius of curvature R1 to the radius of curvature R2. Additionally, in yet other embodiments, the curved surface section 404 may be formed from a plurality of small curved sections each having a small arc length (e.g., 1 degree) and each defined by a constant radius that decreases relative to the anterior-most adjacent small curved section. Additional details regarding various embodiments of the structure of the curved surface section 404 are described further in U.S. Pat. No. 8,828,086 by Williams et al. and entitled “Orthopaedic Femoral Component Having Controlled Condylar Curvature,” U.S. Pat. No. 8,192,498 by Wagner et al. and entitled “Posterior Cruciate-Retaining Orthopaedic Knee Prosthesis Having Controlled Condylar Curvature,” U.S. Pat. No. 8,187,335 by Wyss et al. and entitled “Posterior Stabilized Orthopaedic Knee Prosthesis Having Controlled Condylar Curvature,” and U.S. Pat. No. 8,236,061 by Heldreth et al. and entitled “Orthopaedic Knee Prosthesis Having Controlled Condylar Curvature,” the entirety of each of which is hereby incorporated into the present application by reference.

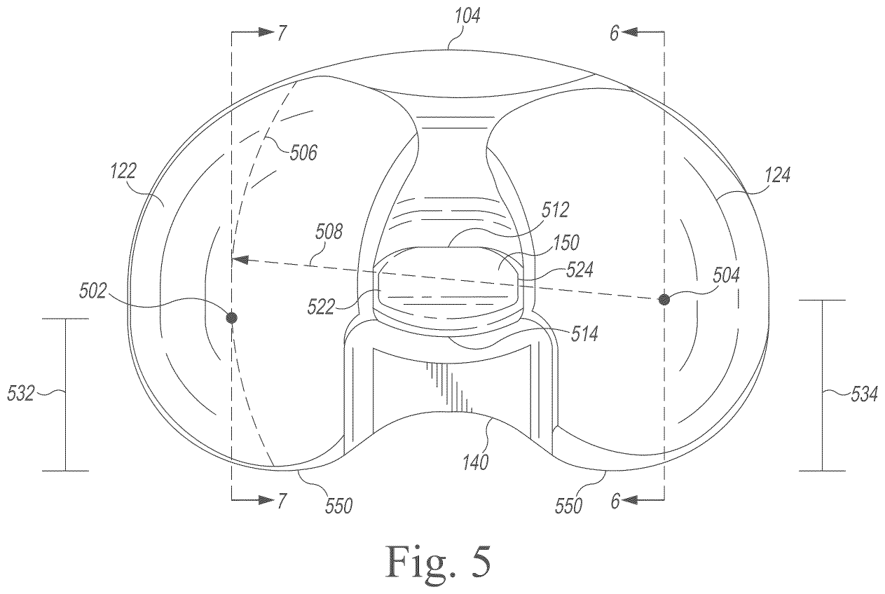

Referring now to FIGS. 5-16, the illustrative tibial insert 104 includes the platform 140, which includes the lateral and medial articular surfaces 122, 124 and the post 150 located therebetweeen as described above. Illustratively, the lateral and medial articular surfaces 122, 124 are asymmetrical to each other. However, in other embodiments, the articular surfaces 122, 124 may be symmetrical. The post 150 includes an anterior surface 512, a posterior surface 514 opposite the anterior surface 512, a lateral sidewall 522 that extends from the posterior surface 514 to the anterior surface 512, and a medial sidewall 524 opposite the lateral sidewall 522 and also extending from the posterior surface 514 to the anterior surface 512.

The medial articular surface 124 of the tibial insert 104 includes a dwell point 504, which defines a distal most point of the medial articular surface 124 and, generally, the contact point or region at which the medial condyle 114 of the femoral component 102 contacts the medial articular surface 124 during articulation (although some contact between the femoral component 102 and the tibial insert 104 may occur anterior to the medial dwell point 504 at some degrees flexion and depending on loading of the femoral component 102 and tibial insert 104). Similarly, the lateral articular surface 122 of the tibial insert 104 includes a dwell point 502, which defines a distal most point of the lateral articular surface 122. Although shown in FIG. 5 and described herein as a “point,” it should be appreciated that the dwell point 502 may be defined as a dwell “region” in other embodiments. That is, the dwell point 502 may be embodied as a region of contact between lateral condyle 112 of the femoral component and the lateral articular surface 122 of the tibial insert 104. Additionally, in some embodiments, such a dwell region may correspond to a semi-planar or semi-flat section of the “sagittal” curvature of the lateral articular surface 122. As used herein, the term “semi-planar” refers to a section that is either planar or is otherwise defined by a radius that is at least three times the length of the radius of curvature of the adjacent curved section(s) as discussed in more detail below. That is, in such embodiments, the lateral dwell region may be embodied as a surface section that is defined by a large enough radius of curvature that the curvature of the dwell region approximates a planar section.

The dwell point 502 (or region) lies on an arcuate articular path 506 of the lateral articular surface 122, which defines a path of contact points between the lateral condyle 112 of the femoral component 102 and the lateral articular surface 122 through flexion of the femoral component 102 (although the lateral condyle 112 may not travel the complete arcuate articular path 506 during normal flexion). The arcuate path 506 is defined by a radius of curvature 508, which has an origin congruent with the dwell point 504 of the medial articular surface 124 or within a reference distance thereof. In the illustrative embodiment, the length of the radius of curvature 808 is design to match, within manufacturing tolerances, the pitch of the condyles 112, 114 of the femoral component 102 (i.e., the distance between the distal-most points on each condyle 112, 114). As such, the curvature of the lateral articular surface 122 is designed to allow the femoral component 102 to pivot or rotate, relative to the medial dwell point 504, along the arcuate articular path 506 during flexion of the femoral component 102. That is, as the femoral component 102 is moved from extension through flexion, the contact point between the lateral condyle 112 of the femoral component 102 and the lateral articular surface 122 moves posteriorly along the arcuate path 506.

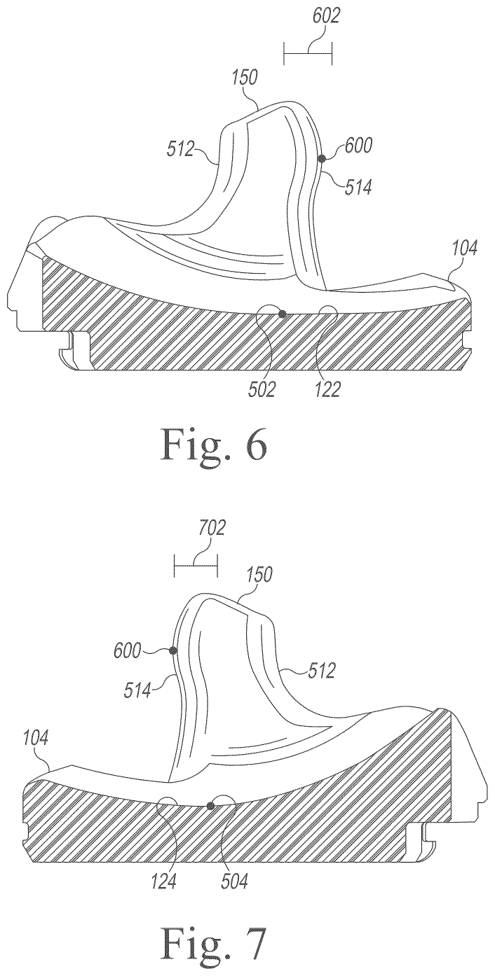

Each of the dwell points 502, 504 are located on the corresponding lateral and medial articular surfaces 122, 124 in a position that promotes or otherwise facilitates the medial pivoting of the femoral component 102 relative to the tibial insert 104 during flexion. For example, in the illustrative embodiment, the lateral dwell point 502 is located more posteriorly relative to the medial dwell point 504. That is, a distance 532 defined between the lateral dwell point 502 and a posterior sidewall 550 of the platform 140 is less than a distance 534 defined between the medial dwell point 504 and the posterior sidewall 550. Additionally, as best shown in FIGS. 6 and 7, each of the dwell points 502, 504 are located anteriorly to a posterior-most point 600 of the posterior surface 514 of the post 150. For example, as shown in FIG. 6, the dwell point 502 of the lateral articular surface 122 is located an anterior-posterior distance 602 anteriorly of the posterior-most point 600 of the posterior surface 514 of the post 150. Similarly, as shown in FIG. 7, the dwell point 504 of the medial articular surface 124 is located an anterior-posterior distance 702 anteriorly of the posterior-most point 600 of the posterior surface 514 of the post 150. In the illustrative embodiments, the distances 602, 702 are not equal due to the lateral dwell point 502 being locate more posteriorly relative to the medial dwell point 504 as discussed above, but may be equal or approximately equal in other embodiments. The size of the distances 602, 702 may be dependent on the particular design criteria and/or size of the tibial insert 104.

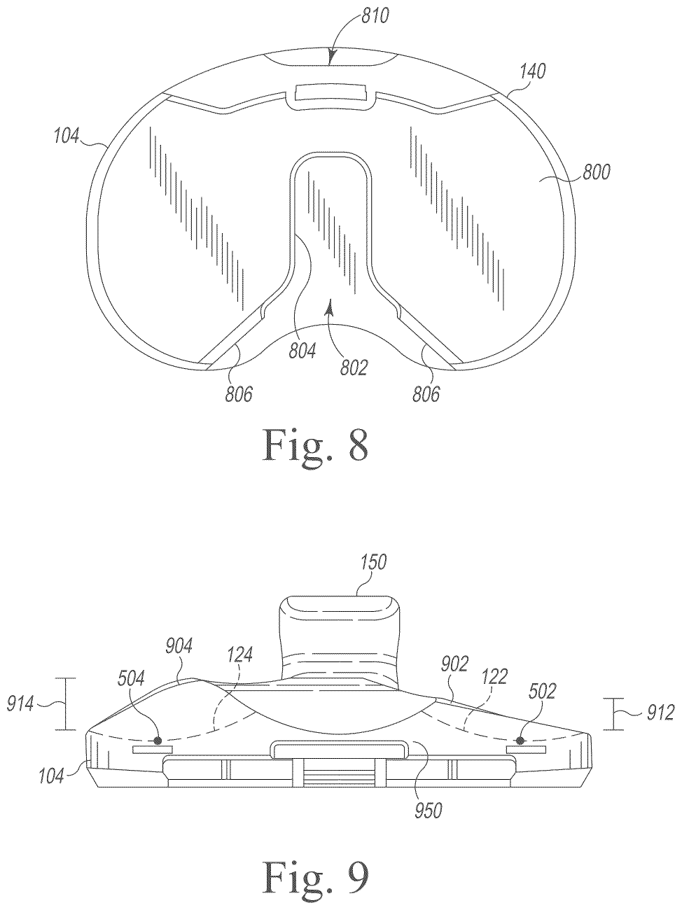

As shown in FIG. 8, the platform 140 of the tibial insert 104 includes a bottom side 800, which is configured to confront a platform of a tibial tray (not shown) during implantation as discussed above. The illustrative tibial insert 104 includes a posterior channel 802 sized and shaped to receive a posterior buttress of the tibial tray. The posterior channel 802 is defined by sidewalls 804, which includes flanges 806 that extend inwardly into the posterior channel 802 and are positioned to be received in undercuts of the corresponding tibial tray. The tibial insert 104 also includes an anterior channel 810 that is sized and shaped to receive an anterior buttress of the corresponding tibial tray. In this way, the channels 802, 810 cooperate with features of the corresponding tibial tray to form a locking mechanism to lock the tibial insert 104 onto the tibial tray in a single orientation relative to the tibial tray. It should be appreciated that the locking mechanism of the tibial insert 104 allows the tibial insert 104 to be used with tibial trays of varying sizes facilitate selection of the a tibial insert size that best matches or cooperates with the selected femoral component 102. Additionally, it should be appreciated that in other embodiments the tibial insert 104 and a corresponding tibial tray may include a mobile bearing interface that allows the tibial insert 104 to move independent of the corresponding tibial tray. Additionally, as discussed above, the tibial insert 104 may be configured to attach directly to the patient's tibia in some embodiments. In such embodiments, the tibial insert 104 may not include the features described above for coupling to a tibial tray, but may include other geometry that allows for implantation of the tibial insert 104 directly onto the patient's bony anatomy (e.g., posts or keels).

As discussed above, the medial articular surface 124 and the lateral articular surface 122 are asymmetric to each other. For example, as shown in FIG. 9, the medial articular surface 124 has an anterior lip 904 that is higher than an anterior lip 902 of the lateral articular surface 122. The anterior lip 902 of the lateral articular surface 122 defines the lip or rim of an anterior sidewall 950 of the platform 140 on the lateral side, and the medial lip 904 of the medial articular surface 124 defines the lip or rim of the anterior sidewall 950 on the medial side. The lateral anterior lip 902 has a lip height 912 defined by a vertical distance (i.e., an inferior-superior distance) between the lateral dwell point/region 502 of the tibial insert 104 and a superior-most point of the lateral anterior lip 902. Similarly, the medial anterior lip 904 has a lip height 914 defined by a vertical distance (i.e., an inferior-superior distance) between the medial dwell point 504 of the tibial insert 104 and the medial anterior lip 904. In the illustrative embodiment, the lip height 914 of the medial anterior lip 904 is greater than the lip height 912 of the lateral anterior lip 902.

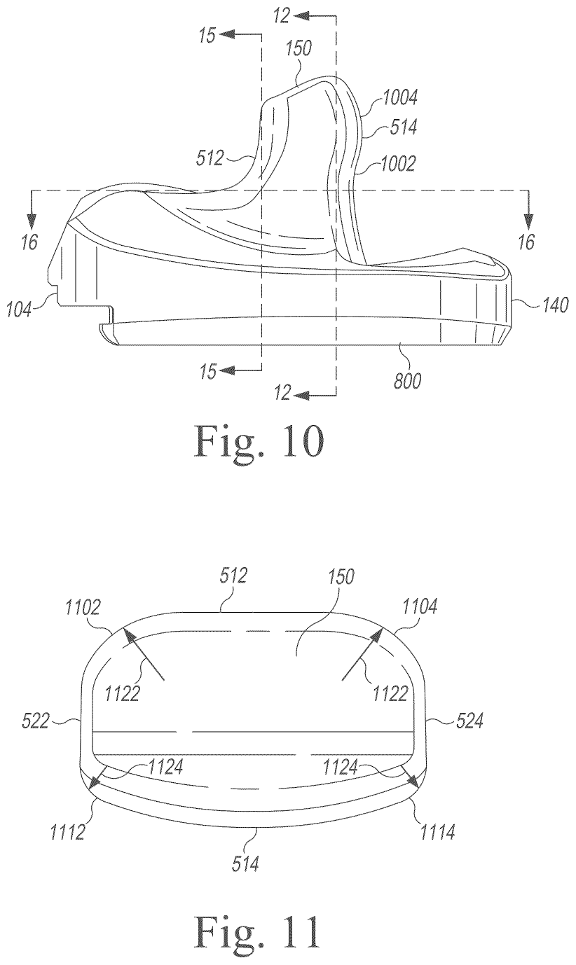

Referring now to FIGS. 10-16 and as discussed above, the post 150 of the tibial insert 104 includes features that promote or otherwise facilitate the medial pivoting of femoral component 102 relative to the tibial insert 104 during flexion. As shown best in FIG. 10, the posterior surface 514 of the post 150 includes a concave section 1002 and a convex section 1004 when viewed in a coronal cross-sectional plane. The concave section 1002 and convex section 1004 cooperate to define an “S-shaped” posterior surface 514, which is configured to contact the posterior cam 130 of the femoral component 102 during a defined range of flexion. The interaction between the posterior cam 130 and the posterior surface 514 of the post 150 promotes rollback of the femoral component 102 during mid and late flexion.

As shown in FIG. 11, the anterior surface 512 of the post 150 and the lateral sidewall 522 cooperate to define or form a lateral-anterior corner 1102 of the post 150, and the anterior surface 512 and the medial sidewall 524 cooperate to define or form a medial-anterior corner 1104 of the post 150. Similarly, the posterior surface 514 of the post 150 and the lateral sidewall 522 cooperate to define or form a lateral-posterior corner 1112 of the post 150, and the posterior surface 514 and the medial sidewall 524 cooperate to define or form a medial-posterior corner 1114 of the post 150. In the illustrative embodiment, each of the anterior corners 1102, 1104 are more “rounded” or gradual relative to the posterior corners 1112, 1114. That is, a radius of curvature 1122 defining the anterior corners 1102, 1104 is greater than a radius of curvature 1124 defining the posterior corners 1112, 1114. The increased “roundness” of the anterior corners 1102, 1104 facilitate the medial pivoting of the femoral component 102 on the tibial insert 104 by providing additional room for the femoral component 102 on the anterior side of the post 150. It should be appreciated that, in other embodiments, only the lateral-anterior corner 1102 may be more rounded relative to the other corners 1104, 1112, 1114 to facilitate the movement of the lateral condyle 112 along the arcuate articular path 506 while the medial condyle 114 remains, generally, stationary at the medial dwell point 504 of the medial articular surface 124.

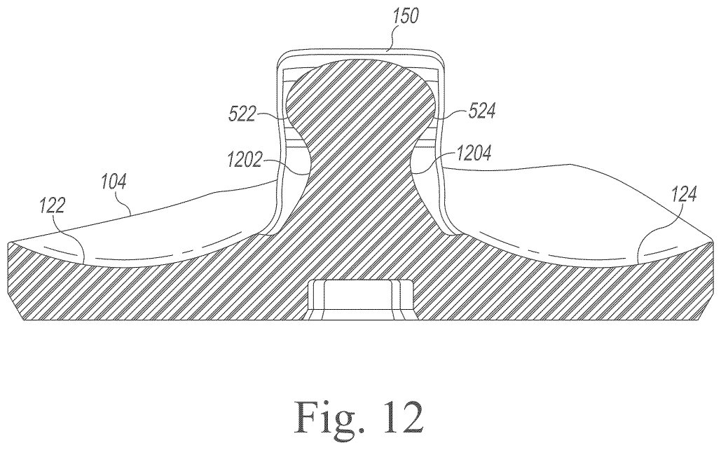

Additionally, as shown best in FIG. 12, each of the lateral sidewall 522 and medial sidewall 524 are concave in the inferior-superior direction. That is, when viewed in a coronal cross-sectional plane as shown in FIG. 12, the lateral sidewall 522 includes a concave curved section 1202 and the medial sidewall 524 includes a concave curved section 1204 such that the post has a substantially “hourglass” shape. It should be appreciated that the “hourglass” shape of the sidewalls 522, 524 may provide an amount of varus/valgus constraint while reducing stress across the fixation surface of the between the femoral competent 102 and tibial insert 104 relative to a post having straight or vertical sidewalls.

Additionally, in some embodiments, the sidewalls 522, 524 may also be concave in the posterior-anterior direction. That is, in such embodiments, each of the sidewalls 522, 524 include a concave section when the post 150 is viewed in a transverse plane. Additionally, the concavity of the concave curved sections 1202, 1204 may be similar or different. For example, in some embodiments, the lateral sidewall 522 may be curved in the inferior-superior direction while the medial sidewall 524 is not.

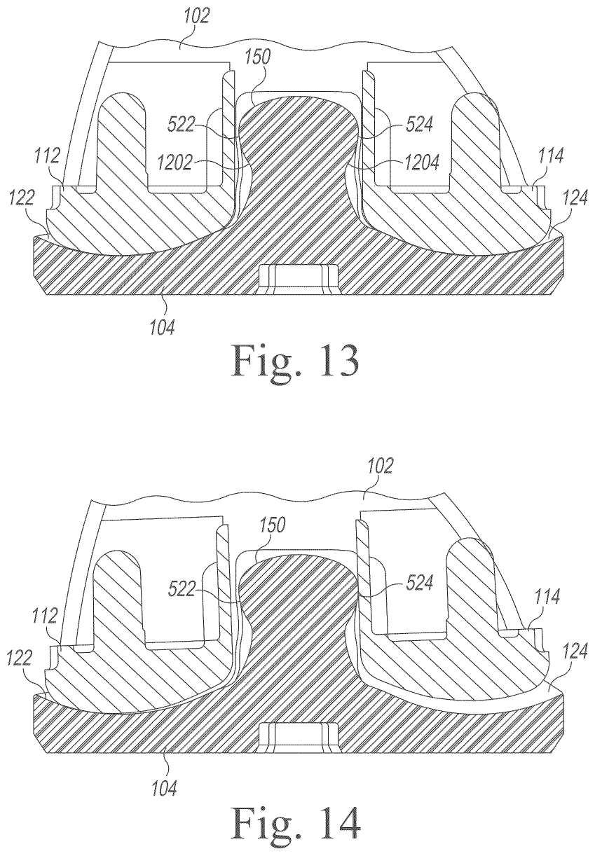

The concavity of the lateral and medial sidewalls 522, 524 facilitate the medial pivoting of the femoral component 102 on the tibial insert 104 by providing additional room for the femoral component 102 on the sidewalls 522, 524 of the post 150. Additionally, the concavity of the sidewalls 522, 524 limit or otherwise restricts lift-off of the femoral component 102 from the tibial insert 104. For example, as shown in FIG. 13, the femoral component 102 is fully seated on the tibial insert 104 such that the lateral condyle 112 of the femoral component 102 is in contact with the lateral articular surface 122 of the tibial insert 104 and the medial condyle 114 of the femoral component 102 is in contact with the medial articular surface 124 of the tibial insert 104. However, during use, the femoral component 102 may exhibit some amount of lift-off from the tibial insert 104. In such cases, the concave shape of the post 150 promotes contact between the inner walls of the lifted condyle 112, 114 of the femoral component and the post 150 as shown in FIG. 14. In the illustrative embodiment, the sidewalls 522, 524 of the post 150 are configured such that a condyle 112, 114 of the femoral component 102 contacts the post 150 at around 1.9 degrees of varus or valgus lift-off to thereby limit or reduce the likelihood of further liftoff of the femoral component 102.

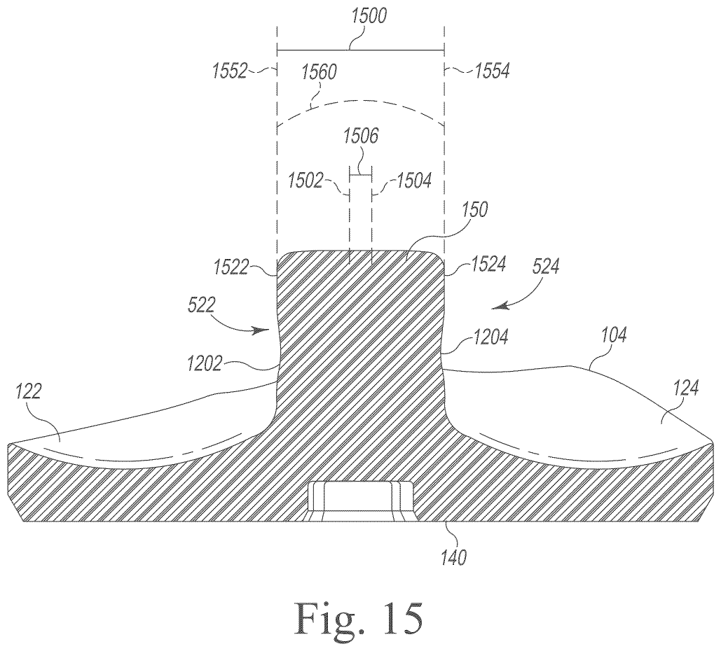

As shown in FIG. 15, the post 150 of the tibial insert 104 has a thickness 1500 measured from the medial-most point and lateral most point of the post 150 that is larger than posts of typical posterior-stabilized tibial inserts. For example, in an illustrative embodiment, the post 150 has a thickness of about 16.2 millimeters for a size 5 tibial insert, relative to 13.8 millimeters of a typical post of a typical size 5 tibial insert. Additionally, in the illustrative embodiment, the post 150 is offset relative to a center plane of the platform 140 of the tibial insert 104. That is, as shown in FIG. 15, a medial-lateral bisecting plane 1502 of the post 150 (i.e., a plane bisecting the post into medial and lateral halves) is offset from a medial-lateral bisecting plane 1504 of the platform 140 of the tibial insert 104. In the illustrative embodiment, the post 150 is offset from the medial-lateral bisecting plane 1504 by a distance 1506 of about 0.02 millimeters. However, in other embodiments, the post 150 may not be offset relative to the center plan e of the platform 140 of the tibial insert 104.

Additionally, in the illustrative embodiment, the lateral sidewall 522 is angled outwardly in the inferior-superior direction releative to the medial sidewall 524. That is, as shown in FIG. 15, the lateral sidewall 522 includes an upper end 1522 located superiorly of the concave section 1202 and the medial sidewall 524 includes an upper end 1524 located superiorly of the concave section 1204. The upper end 1524 of the medial sidewall 524 is substantially planar and defines a vertical plane 1554, while the upper end 1522 of the lateral sidewall 522 is also substantially planer and defines a plane 1552 that is angled relative to the vertical plane 1554 defined by the upper end 1524 of the medial sidewall 524. That is, an angle 1560 of greater than 0 degrees is defined between the plane 1552 defined by the upper end 1522 of the lateral sidewall 522 and the vertical plane 1554 defined by the upper end 1524 of the medial sidewall 524.

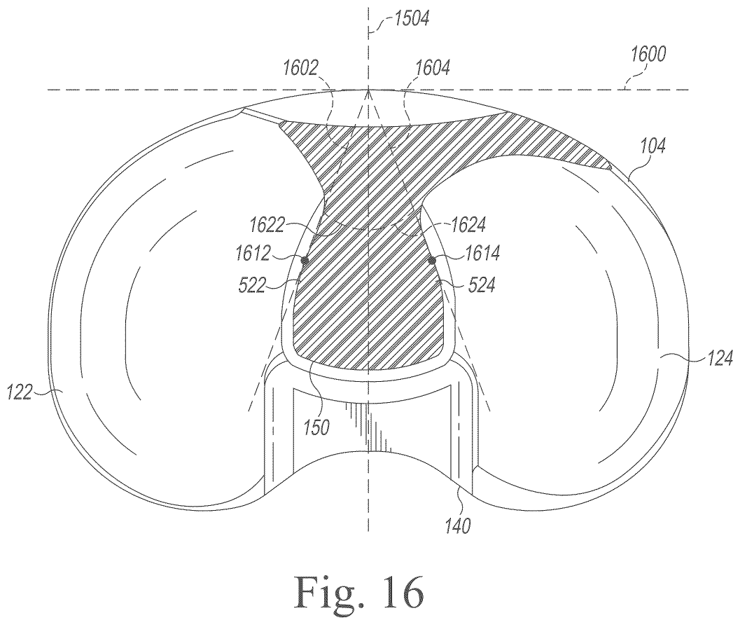

As shown best in the cross-sectional view of FIG. 16 taken generally at the superior-most point of the anterior lip 902 of the lateral articular surface 122 (see FIG. 9), each of the lateral and medial sidewalls 522, 524 slope inwardly in the posterior-anterior direction. However, the lateral sidewall 522 slopes inwardly at a greater rate than the medial sidewall 524 such that additional room for femoral component 102 is provided on the lateral articular surface 122 relative to the medial articular surface 124. That is, the inward slope of the lateral sidewall 522 is “greater” (e.g., relative to a medial-lateral reference axis 1600) than the slope of the medial sidewall 524. For example, as illustrated in FIG. 16, a plane 1602 tangent to a lateral point 1612 on the lateral sidewall 522 of the post 150 defines an angle 1622 relative to the medial-lateral bisecting plane 1504 of the platform 140. Similarly, a plane 1604 tangent to a medial point 1614 on the medial sidewall 524 of the post 150 defines an angle 1624 relative to the medial-lateral bisecting plane 1504 of the platform 140. In the illustrative embodiment, the angle 1622 defined between the plane 1602 and the medial-lateral bisecting plane 1504 is smaller than the angle 1624 defined between the plane 1504 and the medial-lateral bisecting plane 1504 such that the lateral sidewall 522 tapers inwardly at a greater rate or amount than the medial sidewall 524.

Referring now to FIGS. 17-22, as discussed above, the femoral component 102 is configured to articulate on the tibial insert 104 through a range of degrees of flexion. For example, the femoral component 102 is shown at extension (i.e., 0 degrees of flexion) in FIG. 17 and a contact point 1700 between the lateral condyle 112 of the femoral component 102 and the lateral articular surface 122 of the tibial insert 104 is approximately located at the dwell point 502 of the lateral articular surface 122. Additionally, the posterior cam 130 of the femoral component 102 is not in contact with the posterior surface 514 of the post 150.

In FIG. 18, the femoral component 102 has articulated to about 45 degrees of flexion. At that degree of flexion, the contact point 1700 between the lateral condyle 112 of the femoral component 102 and the lateral articular surface 122 of the tibial insert 104 remains approximately located at the dwell point 502 of the lateral articular surface 122. Additionally, the posterior cam 130 of the femoral component 102 is not yet in contact with the posterior surface 514 of the post 150.

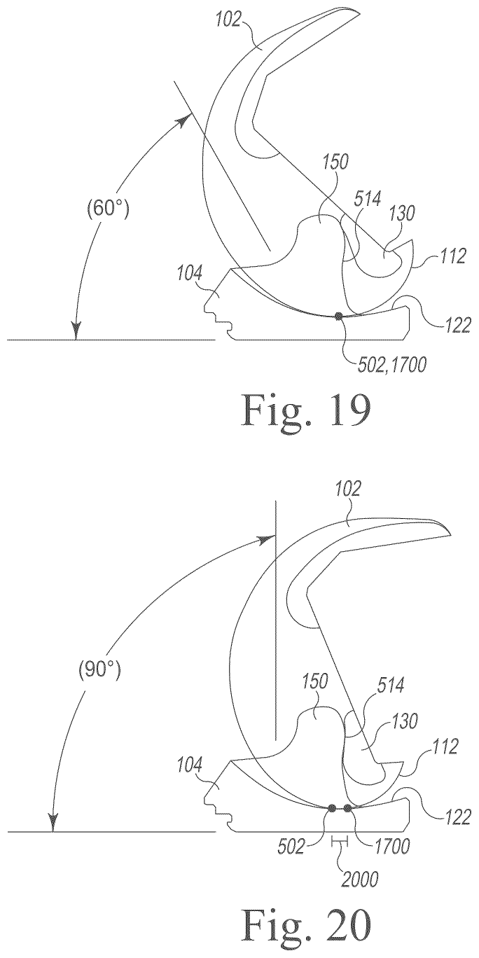

In FIG. 19, the femoral component 102 has articulated further to about 60 degrees of flexion. Again, at that degree of flexion, the contact point 1700 between the lateral condyle 112 of the femoral component 102 and the lateral articular surface 122 of the tibial insert 104 remains approximately located at the dwell point 502 of the lateral articular surface 122. However, the posterior cam 130 of the femoral component 102 has initiated contact with the posterior surface 514 of the post 150, which begins to promote rollback of the femoral component 102.

Subsequently, in FIG. 20, the femoral component 102 has articulated to about 90 degrees of flexion. At that degree of flexion, the contact point 1700 between the lateral condyle 112 of the femoral component 102 and the lateral articular surface 122 of the tibial insert 104 has moved posteriorly from the dwell point 502 of the lateral articular surface 122. For example, in the illustrative embodiment, the contact point 1700 is located a distance 2000 of about 0.8 millimeters posterior of the dwell point 502 of the lateral articular surface 122 when the femoral component is positioned at about 90 degrees of flexion. Additionally, the posterior cam 130 of the femoral component 102 has fully contacted the posterior surface 514 of the post 150.

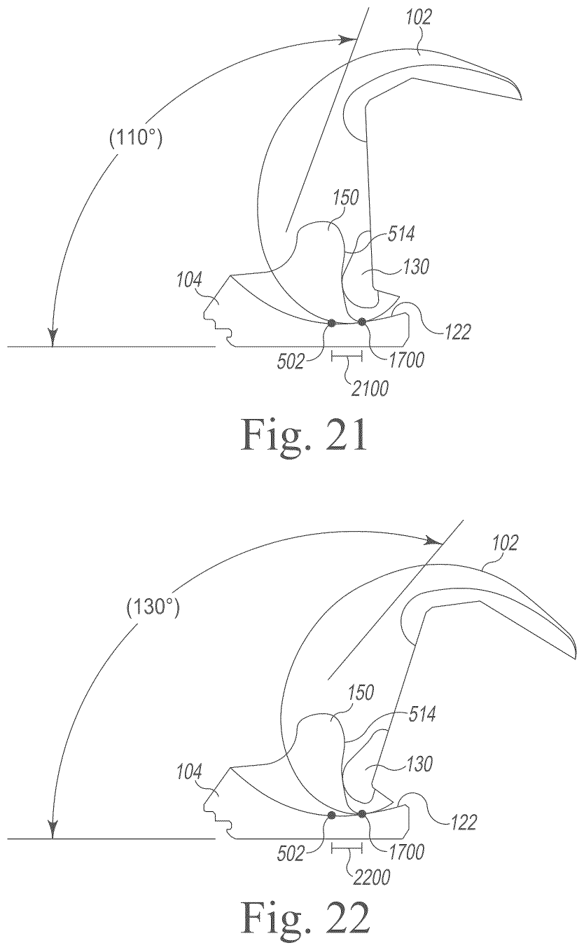

In FIG. 21, the femoral component 102 has articulated further to about 110 degrees of flexion, and the contact point 1700 between the lateral condyle 112 of the femoral component 102 and the lateral articular surface 122 of the tibial insert 104 has moved more posteriorly from the dwell point 502 of the lateral articular surface 122. For example, in the illustrative embodiment, the contact point 1700 is located a distance 2100 of about 3.0 millimeters posterior of the dwell point 502 of the lateral articular surface 122 when the femoral component is positioned at about 110 degrees of flexion. Additionally, the posterior cam 130 of the femoral component 102 remains in contact with the posterior surface 514 of the post 150.

In FIG. 22, the femoral component 102 is shown in deep flexion at an angle of about 130 degrees of flexion. At that degree of flexion, the contact point 1700 between the lateral condyle 112 of the femoral component 102 and the lateral articular surface 122 of the tibial insert 104 has moved even more posteriorly from the dwell point 502 of the lateral articular surface 122. For example, in the illustrative embodiment, the contact point 1700 is located a distance 2200 of about 5.2 millimeters posterior of the dwell point 502 of the lateral articular surface 122 when the femoral component is positioned at about 130 degrees of flexion. Additionally, the posterior cam 130 of the femoral component 102 remains in contact with the posterior surface 514 of the post 150 at that degree of flexion.

As such, it should be appreciated that the illustrative tibial insert 104 includes various features that facilitate or promote the medial pivoting of the femoral component 102 while providing an amount of varus-valgus stability. Additionally, in some embodiments, the tibial insert 104 may be sized or otherwise configured for use with a primary femoral component. In such embodiments, the tibial insert 104 may, for example, replace a primary tibial insert 104 while being configured to properly articulate with the primary femoral component such that the primary femoral component need not be replaced. In other embodiments, the tibial insert 104 may be embodied as, or the features of the tibial insert 104 described above may be embodied in, a primary tibial insert.

Furthermore, it should be appreciated that the illustrative tibial insert 104 may include some, but not all, of the features described above in some embodiments. For example, in some embodiments, the post 150 of the tibial insert 104 may not have an “S-shaped” posterior surface 514. In such embodiments, the post 150 may have a substantially flat or planar posterior surface 514, have a concave posterior surface 514, have a convex posterior surface 514, or have a posterior surface 514 that is partially concave, convex, or otherwise have another geometric shape other than a general “S-shape” as described above. Additionally, in some embodiments, the lateral sidewall 522 and/or the medial sidewall 524 may not include the concave curved sections 1202, 1204, respectively. That is, the sidewalls 522, 524 may not have the “hourglass” shape in some embodiments. In such embodiments, the sidewalls 522, 524 may have a substantially planar coronal cross-section or other geometric shape that provides an amount of varus/valgus constraint. Furthermore, it should be appreciated that sidewalls 522, 524 may be curved in one or more cross-sectional planes. For example, one or both of the sidewalls 522, 524 may be concavely curved when viewed in both a coronal and a transverse plane or in only a coronal plane or only a transverse plane. Additionally, each of the sidewalls 522, 524 may be identically curved or may have a curved shape different from each other.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and/or systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. An orthopaedic insert comprising:
a platform having a lateral articular surface configured to articulate with a lateral condyle of a femoral component and a medial articular surface configured to articulate with a medial condyle of the femoral component, wherein the medial articular surface is asymmetrically shaped relative to the lateral articular surface; and
a post extending superiorly from the platform and located between the lateral and medial articular surfaces, wherein the post has a posterior surface, an anterior surface opposite the posterior surface, a lateral sidewall extending from the posterior surface to the anterior surface, and a medial sidewall opposite the lateral sidewall and extending from the posterior surface to the anterior surface,
wherein the medial and lateral side walls of the post are concave when viewed in a coronal cross-sectional plane.

2. The orthopaedic insert of claim 1, wherein a medial-lateral bisecting plane of the post is offset from a medial-lateral bisecting plane of the platform.

3. The orthopaedic insert of claim 2, wherein the medial-lateral bisecting plane of the post is offset from the medial-lateral bisecting plane of the platform in the lateral direction.

4. The orthopaedic insert of claim 1, wherein the medial and lateral side walls of the post are also concave when viewed in a sagittal cross-section plane.

5. The orthopaedic insert of claim 1, wherein each of the lateral and medial side walls includes a concave section and an upper end located superiorly to the respective concave section, and
wherein the upper end of the medial sidewall defines a vertical plane and the upper end of the lateral sidewall is angled relative to the vertical plane defined by the medial sidewall.

6. The orthopaedic insert of claim 1, wherein an angle defined between a medial-lateral bisecting plane of the platform and a first plane tangent to a first point located on the lateral side wall is less than an angle defined by the medial-lateral bisecting plane of the platform and a second plane tangent to a second point located on the medial sidewall, wherein the first point and second point are equidistant from a bottom surface of the platform.

7. The orthopaedic insert of claim 1, wherein the lateral articular surface includes a lateral dwell point that defines a distal-most point on the lateral articular surface and the medial articular surface includes a medial dwell point that defines a distal-most point on the medial articular surface, and
wherein a posterior-most point on the posterior surface of the post is located posteriorly of the lateral dwell point and of the medial dwell point.

8. The orthopaedic insert of claim 1, wherein:
the lateral articular surface includes an anterior lateral lip and a lateral dwell point that defines a distal-most point on the lateral articular surface, and wherein an inferior-superior distance between the lateral dwell point and a superior-most point of the anterior lateral lip defines a lip height of the anterior lateral lip, and
the medial articular surface includes an anterior medial lip and a medial dwell point that defines a distal-most point on the medial articular surface, and wherein an inferior-superior distance between the medial dwell point and a superior-most point of the anterior medial lip defines a lip height of the anterior medial lip,
wherein the lip height of the anterior medial lip is greater than the lip height of the anterior lateral lip.

9. The orthopaedic insert of claim 1, wherein the posterior surface and each of the lateral sidewall and the medial sidewall forms a pair of posterior corners of the post and wherein the anterior surface and each of the lateral sidewall and the medial sidewall forms a pair of anterior corners of the post,
wherein the anterior corners have a greater radius of curvature than the posterior corners.

10. The orthopaedic insert of claim 1, wherein the posterior surface of the post has an “S-shaped” coronal cross-section that includes a concave section and a convex section that is superior to the concave section.

11. The orthopaedic insert of claim 1, wherein the medial and lateral side walls are also concave when viewed in a transverse cross-sectional plane that bisects the post.

12. An orthopaedic knee prosthesis comprising:
a tibial insert including (i) a platform having a lateral articular surface configured to articulate with a lateral condyle of a femoral component and a medial articular surface configured to articulate with a medial condyle of the femoral component, wherein the medial articular surface is asymmetrically shaped relative to the lateral articular surface and (ii) a post that extends superiorly from the platform and is located between the lateral and medial articular surfaces, wherein an angle defined between a medial-lateral bisecting plane of the platform and a first plane tangent to a first point located on the lateral side wall is less than an angle defined by the medial-lateral bisecting plane of the platform and a second plane tangent to a second point located on the medial sidewall, wherein the first point and second point are equidistant from a bottom surface of the platform.

13. The orthopaedic knee prosthesis of claim 12, further comprising a femoral component having a lateral condyle and a medial condyle, wherein each of the lateral condyle and the medial condyle includes a femoral articular surface defined by a plurality of curved femoral surface sections that includes a first curved femoral surface section defined by a continually decreasing radius of curvature.

14. The orthopaedic knee prosthesis of claim 13, wherein the lateral articular surface of the tibial insert includes a lateral dwell point that defines a distal-most point on the lateral articular surface, and
wherein a contact point between the femoral articular surface of the lateral condyle of the femoral component and the lateral articular surface of the tibial insert lies on the lateral dwell point of the lateral articular surface of the tibial insert when the femoral component is positioned in 0 degrees of flexion.

15. The orthopaedic knee prosthesis of claim 14, wherein the contact point between the femoral articular surface of the lateral condyle of the femoral component and the lateral articular surface of the tibial insert moves posteriorly relative to the lateral dwell point of the lateral articular surface of the tibial insert as the femoral component is moved through a range of flexion.

16. The orthopaedic knee prosthesis of claim 14, wherein the contact point between the femoral articular surface of the lateral condyle of the femoral component and the lateral articular surface of the tibial insert is located posteriorly of the lateral dwell point of the lateral articular surface of the tibial insert by more than 0.5 millimeters when the femoral component is positioned at 90 degrees of flexion.

17. The orthopaedic knee prosthesis of claim 12, wherein a medial-lateral bisecting plane of the post is offset from a medial-lateral bisecting plane of the platform.

18. The orthopaedic knee prosthesis of claim 12, wherein each of the lateral and the medial side walls includes a concave section and an upper end located superiorly to the respective concave section, and
wherein the upper end of the medial sidewall defines a vertical plane and the upper end of the lateral sidewall is angled relative to the vertical plane defined by the medial sidewall.

19. The orthopaedic knee prosthesis of claim 12, wherein the lateral articular surface includes a lateral dwell point that defines a distal-most point on the lateral articular surface and the medial articular surface includes a medial dwell point that defines a distal-most point on the lateral articular surface, and
wherein a posterior-most point on the posterior surface of the post is located posteriorly of the lateral dwell point and of the medial dwell point.

20. The posterior-stabilized orthopaedic knee prosthesis of claim 12, wherein:
the lateral articular surface includes an anterior lateral lip and a lateral dwell point that defines a distal-most point on the lateral articular surface, and wherein an inferior-superior distance between the lateral dwell point and a superior-most point of the anterior lateral lip defines a lip height of the anterior lateral lip, and
the medial articular surface includes an anterior medial lip and a medial dwell point that defines a distal-most point on the medial articular surface, and wherein an inferior-superior distance between the medial dwell point and a superior-most point of the anterior medial lip defines a lip height of the anterior medial lip,
wherein the lip height of the anterior medial lip is greater than the lip height of the anterior lateral lip.
